(19) United States
(12) Patent Application Publication
(10) Pub. No.: US 2020/0088721 A1
(43) Pub. Date: Mar. 19, 2020

(54) Glycoprotein assay method

(71) Applicant: J-OIL MILLS, INC., Tokyo (JP)

(72) Inventors: Hiroto MATSUI, Tokyo (JP); Yuka KOBAYASHI, Tokyo (JP); Yasushi UENO, Tokyo (JP)

(21) Appl. No.: 16/070,752

(22) PCT Filed: Jan. 25, 2017
(86) PCT No.: PCT/JP2017/002516

§ 371 Date: Jul. 17, 2018

(30) Foreign Application Priority Data
Jan. 27, 2016 (JP) 2016-013187

(51) Int. Cl. G01N 33/548

(52) U.S. Cl. CPC G01N 33/548

(57) ABSTRACT

[Problem] To provide a method for improving the detection sensitivity (S/N ratio) for conjugates of a glycoprotein and a sugar-binding compound in order to detect glycoproteins with a high degree of precision using a sugar-binding compound.

[Solution] The glycoprotein assay method according to the present invention comprises reacting a glycoprotein with a sugar-binding compound having affinity with a glycan contained in the glycoprotein to detect the reacted sugar-binding compound, wherein a pH level is adjusted to an alkaline pH range of more than 8.5 to less than 11.0, in at least one step selected from among a group of steps consisting of the reaction step of the glycoprotein with the sugar-binding compound, and treatment steps subsequent thereto. The sugar-binding compound is preferably a sugar-binding protein.

### TECHNICAL FIELD

The present invention relates to a glycoprotein assay method, more specifically a method for improving an S/N ratio during measurement of glycoprotein,

### BACKGROUND ART

More than half of proteins are glycosylated after translation. The manners of glycans binding to proteins are classified into an N-linked type in which a glycan binds to an amide group of an asparagine residue and an O-linked type in which a glycan hinds to a hydroxyl group of a serine or threonine residue. Both glycosylations play important roles in protein activity, cell-cell interaction, adhesion and the like. There have been a lot of reports that change in glycosylation is associated with diseases.

For example, an α-fetoprotein is a glycoprotein contained in serum having an N-linked glycan and scarcely exists in serum of healthy adults. On the other hand, in serum of a patient with benign liver disease, an α-fetoprotein-L1 type glycan (AFP-Li) increases, and furthermore, in a patient with liver cancer, a fucosylated α-fetoprotein-L3 type glycan (fAFP or AFP-13) is detected. Difference in the glycan detected with a lectin has been used for diagnosing liver diseases.

Haptoglobin is a glycoprotein having four N-linked glycan binding sites on a β chain (molecular weight: 40,000). In pancreatic cancer, a lesional haptoglobin generated by addition of a fucose to haptoglobin is detected from serum and the like of a patient. The lesional haptoglobin increases with stage progression of pancreatic cancer and disappears after removal of a tumor part of pancreatic cancer. Early detection of pancreatic cancer is expected by precise and rapid detection of the fucosylated haptoglobin.

Thyroglobulin is a hormone that is synthesized in epithelial cells of a thyroid gland and accumulates in follicles, and generally has an activity of acting on cells all over the body to increase a cellular metabolic rate. A representative example of hyperthyroidism with excessively secreted thyroid hormone is Basedow's disease. Basedow's disease causes symptoms such as limb tremor, exophthalmos, palpitations, thyrocele, hyperhidrosis, weight loss, hyperglycemia and hypertension. Chronic thyroiditis (Hashimoto's disease) is an example of hypothyroidism with deficient thyroid hormone. Hashimoto's disease causes symptoms such as general malaise, hypohidrosis, weight gain and constipation. This protein has fucose as a glycan. The measurement accuracy for the thyroglobulin content can be improved by enhancing detection sensitivity of a glycan added to a thyroglobulin.

In a patient with rheumatoid arthritis, an addition rate of terminal galactoses in serum IgG decreases and a rate of glycans having N-acetylglucosamine on their terminals increases. Galactose deletion markedly impairs important physiological functions of activation of complements and ability of binding to Fc receptors.

Transferrin (TF) is a glycoprotein consisting of a polypeptide chain having 679 amino acids, in which the 413rd and 611st aspartic acid residues are N-glycosylated with two branched glycans having sialic acids at terminals. The transferrin includes polymorphisms of TFC1 in which the 570th amino acid residue is proline and TFC2 in which the prolific is substituted with serine. In a patient with Alzheimer disease (AD) having a TFC1C2 heterozygotic genotype, a relative intensity of a TF having 6 sialic acids significantly decreases compared to that of patients having a TFC1C1 homozygotic genotype.

In a CSF glycoprotein collected from an AD patient, an addition rate of sialic acid significantly decreases. Changes in the amount of sialic acid have been observed for cardiovascular diseases, alcoholism, diabetes and the like, in addition to AD.

For detection of the glycoproteins, the use of a lectin that is one kind of sugar-binding compounds is known. The lectin is a generic name of proteins showing affinity with sugar residues such as sialic acid, galactose and N-acetylglucosamine. A large number of lectins derived from plants, animals or fungi having affinity with specific sugar residues have been discovered.

Enzyme immunoassay (lectin ELISA) is known as a method for detecting a glycoprotein using a lectin. The lectin ELISA has advantages such as an ability of simultaneously measuring a large number of specimens and an ability of relatively easily measuring glycans.

### PRIOR ART DOCUMENTS

### Non-Patent Documents

Non-patent Document 1: Yuka Kobayashi et al., “A Novel Core Fucose-specific Lectin from the Mushroom Pholiota squarrosa”, J. Biol. Chem, 2012, 287, p33973-33982

### SUMMARY OF INVENTION

### Problem to be Solved

In the glycoprotein assay method such as the conventional lectin ELISA, noises derived from a sample (e.g. serum) containing a glycoprotein are generated. If the detection sensitivity (S/N ratio) during detection of the glycoprotein is love, it is difficult to precisely detect the glycoprotein. An improved SIN ratio of the glycoprotein assay method such as the lectin ELISA is desired for early and precisely diagnosing a disease associated with change in an amount of glycans.

Thus, an object of the present invention is to provide a method for improving the detection sensitivity (S/N ratio) for a conjugate of a glycoprotein and a sugar-binding compound in order to precisely detect glycoproteins by a sugar-binding compound including lectins.

### Solution to Problem

As a result of intensive studies on the above problems, the present inventors have found that the above problems can be solved by adjusting a pH level in a specific step in a glycoprotein assay method by a reaction of a glycoprotein with a sugar-binding compound to an alkaline pH range. That is, the present invention provides a glycoprotein assay method, comprising reacting a glycoprotein with a sugar-binding compound having affinity with a glycan contained in the glycoprotein to detect the reacted sugar-binding compound, wherein the pH level is adjusted to an alkaline pH range of more than 8.5 to less than 11.0, in at least one step selected from among the group of steps consisting of the reaction step of the glycoprotein with the sugar-binding compound, and the treatment steps subsequent thereto.

The term “glycoprotein” is herein used to include a glycopeptide. The term “glycan” is herein used to include a monosaccharide. In addition, the “sugar-binding compound” herein means a compound capable of binding to a sugar. The “sugar-binding compound” is preferably a sugar-binding protein.)

It is preferable that the method essentially comprises adjusting the pH level in the reaction step of the glycoprotein with the sugar-binding compound to the alkaline pH range.

Preferably, the glycoprotein is immobilized to a support.

Preferably, the glycoprotein is immobilized to the support via its antibody

Preferably, the sugar-binding compound and/or a probe for detecting the sugar-binding compound are labeled.

The glycan is a complex-type glycan or an O-linked glycan, for example.

The glycoprotein is one selected from a group consisting of haptoglobin (HP), fucosylated haptoglobin, transferrin (TF), γ-glutamyltranspeptidase (γ-GTP), immunoglobulin G (IgG), immunoglobulin A (IgA), immunoglobulin M (IgM), α1-acidic glycoprotein (AGP), α-fetoprotein (AFP), fucosylated α-fetoprotein (fAFP, AFP-L3), fibrinogen, human placenta chorionic gonadotropin (hCG), carcinoembryonic antigen (CEA), prostate-specific antigen (PSA), thyroglobulin (TG), fetuin (FET), asialofetuin (aFET) and ovalbumin (OVA), for example.

### Effects of Invention

According to the glycoprotein assay method of the present invention, an S/N ratio during the detection of glycoproteins is improved compared to the conventional methods. In particular, the pH levels in the reaction step of the glycoprotein with the sugar-binding compound and/or in the probe reaction step of the sugar-binding compound in a conjugate of the glycoprotein and the sugar-binding compound with a secondary probe are adjusted to the above-described specific pH range, so that the S/N ratio is markedly improved. When deletion or addition of a glycan from a glycoprotein is associated with a disease, an improved S/N ratio during the detection of glycoproteins leads to early detection, diagnosis and treatment of the disease. Also, it is expected to be useful in elucidation of pathogenic mechanisms of diseases, and medical and biochemical studies on treatment and prevention.

### BRIEF DESCRIPTION OF DRAWINGS

FIG. 1a illustrates a graph comparing signals and noises when changing conditions in a process that a glycoprotein TG is solidified on an ELISA plate, then blocked, to which serum is added to non-specifically adsorb noise sources, and with which subsequently a Pholiota squarrosa lectin (PhoSL) solution is reacted. In Comparative Example 1, a lectin reaction was carried out in a PBS at pH 7.4. In Examples 1 to 3, lectin reactions were carried out in three buffers at pH 10.0. In Examples 1 to 3, noises remarkably decreased. On the other hand, signals were maintained at the level in Comparative Example 1.

FIG. 1b illustrates a graph comparing the S/N ratios in FIG. 1a. The S/N ratios in Examples 1 to 3 in which the lectin reactions were carried out at pH 10.0 remarkably increased compared to that in Comparative Example 1 in which the lectin reaction was carried out at pH 7.4.

FIG. 2a illustrates a graph showing S/N ratios when changing the pH adjustment during the lectin reaction (primacy reaction) in FIG. 1a into pH adjustments after the specimen reaction (i.e. before primary reaction) and during washing after the primary reaction or secondary reaction. The S/N ratio was not improved by the pH adjustment during alkaline washing before the primary reaction. On the other hand, the improved S/N ratio due to the noise reducing effect was confirmed by washing at an alkaline pH level after the primary reaction or the secondary reaction.

FIG. 2b illustrates a graph showing S/N ratios when changing the pH adjustment of the reaction solution during the lectin reaction (primary reaction) in FIG. 1a into pH adjustments of a reaction solution during the secondary reaction or reaction solutions during the primary reaction and secondary reaction. It was confirmed that the S/N ratio was improved due to the noise reducing effect similarly to the adjustment during the primary reaction by alkalizing a solvent in the secondary reaction solution, and the effect was further improved by combining the adjustment during the primary reaction.

FIG. 2c illustrates a graph showing S/N ratios when changing the pH adjustment of the reaction solution during the lectin reaction in FIG. 1a (primary reaction) into the pH adjustment of the reaction solution during the secondary reaction and changing the secondary probe into an AP-labeled streptavidin. The improved S/N ratio due to the noise reducing effect was confirmed similarly to the HRP-labeled streptavidin even if the secondary probe was changed into the AP-labeled streptavidin.

FIG. 2d illustrates a graph showing S/N ratios when changing the pH adjustment of the reaction solution during the lectin reaction in FIG. 1a (primary reaction) into the pH adjustment of the reaction solution during the secondary reaction, using a non-labeled PgiSL as the lectin in the primary reaction, using a biotin-labeled anti-PhoSL antibody as the secondary probe, and using an HRP-labeled streptavidin as the tertiary probe. The improved S/N ratio due to the noise reducing effect was confirmed even if using the anti-PhoSL antibody as the secondary probe.

FIG. 3a illustrates a graph showing the relationship between the pH level and the signal/noise when reacting a glycoprotein fAFP (pepsin-treated) with the PhoSL with varying the pH level in a glycine-sodium hydroxide buffer.

FIG. 3b illustrates a graph showing the relationship between the pH level and the signal/noise when reacting a glycoprotein fAFP (pepsin-treated) with the PhoSL with varying the pH level in a carbonate-bicarbonate buffer.

FIG. 3c illustrates a graph showing the relationship between the pH level and the signal/noise when reacting a glycoprotein fAFP (pepsin-treated) with the PhoSL with varying the pH level in a. TAPS buffer.

FIG. 3d illustrates a graph showing the SIN ratios in FIGS. 3a to 3c. This figure indicates that the detection sensitivity for the glycoprotein is improved by adjusting the pH levels of the solvents used during the lectin reaction and the steps subsequent thereto to a level within a range of more than 8.5 to less than 11.0, preferably a range of 8.6 to 10.5, more preferably a range of 9.0 to 10.5,

FIG. 4a illustrates a graph showing signals and noises when detecting the reaction of the glycoprotein fAFP (pepsin-treated) in FIG. 3a with the PhoSL by a microbead lectin ELISA. It is shown that even if the immobilization support is changed to microbeads, the noise reducing effect is exhibited by adopting an alkaline pH level as in the present invention.

FIG. 4b illustrates a graph showing the S/N ratio in FIG. 4a. Also with the microbead ELISA, an improved S/N ratio due to the noise reducing effect was confirmed.

### DESCRIPTION OF EMBODIMENTS

Hereinafter, an embodiment of the present invention will be described in detail. The glycoprotein assay method of the present invention comprises reacting a glycoprotein with a sugar-binding compound having affinity with a glycan in the glycoprotein to detect the reacted sugar-binding compound (glycoprotein-sugar-binding compound conjugate), and essentially comprises adjusting a pH level in at least one step selected from a group consisting of a step of reacting the glycoprotein with the sugar-binding compound and the treatment steps subsequent thereto to an alkaline pH range of more than 8.5 to less than 11.0.

The glycans to be measured in the present invention includes an N-linked glycan and an O-linked glycan. The N-linked glycan includes:

a complex-type glycan in which 1 to 6 side chains composed of fucose, sialic acid, galactose and N-acetylglucosamine (N-acetyllactosamine structure, poly N-acetylgluctosamine structure) are added to a core structure represented by the following formula:

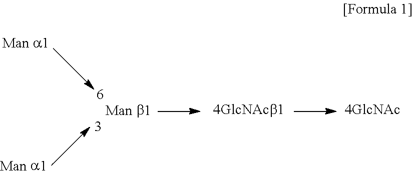

[wherein, Man refers to mannose, and GlcNAc refers to N-acetyllactosamine]:

a high mannose type glycan in which an oligosaccharide composed only of mannose is added to the core structure; and

a hybrid type glycan in which the conjugated type and the high mannose type are mixed.

Also, the N-linked glycan includes a glycan in which fucose is added to the N-acetylglucosamine at the reducing terminal of the core structure.

The glycans to be measured in the present invention include sialic acid (Sia), galactose (Gal), mannose (Man), N-acetylglucosamine (G1cNAc), N-acetylgalactosamine (GalNAc), fucose (Fuc), and the like.

Specific examples of the glycoproteins include haptoglobin (HP), fucosylated haptoglobin (HAP), transferrin (TF), 65 -glutamyltranspeptidase (γ-GTP), immunoglobulin G (IgG), immunoglobulin A (IgA), immunoglobulin M (IgM), al-acidic glycoprotein (AGP), α-fetoprotein (AFP), fucosylated α-fetoprotein (fAFP, AFP-L3), fibrinogen, human placenta chorionic gonadotropin (hCG), carcinoembryonic antigen (CEA), prostate-specific antigen (PSA), thyroglobulin (TG), fetuin (FET), asialofetuin (aFET), ovalbumin (OVA) and the like. A glycoprotein for which a relationship between a change in a glycan structure in the glycoprotein and a disease or abnormality is suggested is preferable.

The origin of the glycoprotein is not particularly limited. The examples thereof include blood, plasma, serum, tear, saliva, body fluid, milk, urine, culture supernatant of cells, secretion from a transgenic animal, and the like. Blood, plasma or serum is preferable, and serum is particularly preferable. If the method of the present invention is applied to a sample of blood, plasma or serum, noises attributed to blood, plasma or serum can be reduced.

The sample to be subjected to the assay method of the present invention may be not only the glycoprotein, but also a fragment (glycopeptide) thereof as long as it has a glycan. The glycopeptide is obtained by treating a glycoprotein with a protease (proteolytic enzyme). The protease is not particularly limited as long as it acts on a glycoprotein to produce a glycopeptide. The proteases can he functionally classified into aspartic protease (acid protease), serine protease, cysteine protease, metalloprotease, N-terminal threonine protease, glutamic protease and the like.

The protease may be originated from anything. Examples of animal-derived proteases include pepsin, trypsin, chymotrypsin, elastase, cathepsin D, calpain, and the like. Examples of the plant-derived proteases include papain, chymopapain, actinidin, kallikrein, ficin, bromelain and the like. Examples of microorganism-derived proteases include those derived from Bacillus, Aspergillus, Rhizopus, blue mold (Penicillium), Streptomyces, Staphylococcus, Clostridium and Lysobacter.

As the protease used in the present invention, a commercial product can be used without particular limitation. The pepsin used in Examples of the present invention includes a swine gastric mucosa-derived pepsin (from Sigma-Aldrich Co. LLC), and the Streptomyces-derived protease includes Actinase E (from Kaken Pharmaceutical Co., Ltd.) and the like.

The protease treatment is normally carried out in an aqueous medium such as water and a buffer. Use of a buffer is preferable for carrying out the protease treatment at a constant pH level. Examples of the buffer include a glycine hydrochloride buffer, phosphate buffered saline (PBS) and the like. A denaturant such as a surfactant may be added to the aqueous medium in order to facilitate the protease treatment.

The amount of the protease for use may be any amount allowing progression of the reaction of the glycoprotein with the protease. The conditions (pH, temperature, and time) for the protease treatment depend on the protease for use.

After the protease treatment, the enzymatic reaction is terminated by an appropriate means such as change of pH, heat treatment and addition of an enzymatic reaction-terminating liquid. Subsequently, the reaction solution may be separated into a supernatant and a solid residue by a separation means such as filtration, dialysis and centrifugation. The supernatant may be further subjected to deproteinization such as salting-out and ethanol precipitation.

In the assay method of the present invention, the glycoprotein does not necessarily need to be immobilized, but it is preferably immobilized. Examples of the support for immobilizing the glycoprotein include a microtiter plate, beads, a disk, a stick, a tube, a microsensor chip, a microarray, and the like which are made of materials such as glass, polyethylene, polypropylene, polyvinyl acetate, polyvinyl chloride, polymethacrylate, latex, agarose, cellulose, dextran, starch, dextrin, silica gel and porous ceramics. As a method for immobilizing glycoproteins on these supports, a general-purpose method such as physical adsorption, covalent binding and crosslinking can be used without particular limitation.

The glycoprotein may be immobilized on a support via its antibody. The antibody may be the antibody molecule itself, or may be an active fragment containing an antigen-recognition site such as Fab, Fab′, F(ab′) 2 obtained by enzymatic treatment of the antibody.

The origin of the antibody is not limited. The antibodies include an antisera and an ascites fluid obtained by immunizing a mammal such as human, mouse and rabbit with a glycoprotein as an antigen, as well as a polyclonal antibody obtained by purifying them by a general-purpose method such as salting-out, gel filtration, ion exchange chromatography, electrophoresis and affinity chromatography. Furthermore, the antibody include a monoclonal antibody obtained by a process that an antibody-producing lymphocyte of a mouse immunized with a protein prepared from a human or animal serum or the like is fused with a myeloma cell to obtain a hybridoma that produces a monoclonal antibody capable of recognizing the glycoprotein, then the hybridoma or a cell line derived therefrom is cultured, and the monoclonal antibody is collected from the culture. For general-purpose glycoproteins, antibodies thereof are sold as reagents, and they can be used without limitation in the present invention.

When the above-described antibody or the like has a glycan of reacting with a sugar-binding compound, the glycan is removed from the antibody as appropriate. Methods for obtaining an antibody having no glycan capable of reacting with a sugar-binding compound include: a method of treating a monoclonal antibody with a glycan degrading enzyme such as neuraminidase, β-galactosidase and N-glycanase; a method of subjecting an Fc portion of an antibody to limited proteolysis with a protease such as pepsin and papain; a method of oxidatively decomposing a glycan structure with a periodic acid aqueous solution; and a method of adding a glycan synthesis inhibitor to a medium of a hybridoma or an animal cell derived from the hybridoma and culturing the medium.

As a method for immobilizing the antibody to the support, general-purpose methods such as physical adsorption, covalent binding and crosslinking can be used without particular limitation. A solution of an antibody against a glycoprotein (e.g. anti-transferrin antibody) is added to the support to bind the antibody to the support.

In the method of the present invention, first, a solution of a specimen containing a glycoprotein (e.g., serum) is added to a support to which the antibody is appropriately bound, so that the glycoprotein is bound to the support.

Next, the glycoprotein-containing solution is reacted with a sugar-binding compound solution having affinity with a glycan in the glycoprotein to react the glycoprotein with the sugar-binding compound. The sugar-binding compound for use is appropriately selected depending on the glycan capable of binding to the glycoprotein.

The sugar-binding compound is e.g., a protein (including peptide) capable of binding to a sugar, as well as a nucleic acid such as DNA and RNA capable of binding to a sugar.

Examples of the sugar-binding protein include lectin, anti-glycan antibody, maltose binding protein, glucose binding protein, galactose binding protein, cellulose binding protein, chitin binding protein and sugar-binding module. The sugar-binding compound is preferably a sugar-binding protein, more preferably lectin and anti-glycan antibody, still more preferably lectin.

The sugar-binding compounds may be used either alone or in combination of two or more kinds.

When the affinity of the lectin is expressed as the minimuminhibitory concentration of the sugar capable of inhibiting hemagglutination, it is normally 100 mM or less, preferably 10 mM or less. The minimum inhibitory concentration means the minimum concentration required for the sugar to inhibit the agglutination, indicating that the less the minimum inhibitory concentration is, the more the affinity with the lectin is. The hemagglutination inhibition test method can be carried out in accordance with the method described in Japanese Patent Nor 4514163 (fucose specific lectin).

The lectin may be either a naturally-derived lectin or a lectin obtained by chemical synthesis or genetic engineering synthesis. The lectin may be originated from any of a plant, an animal and a fungus. Examples of natural lectins that can be used in the present invention are shown below.

Examples of lectins having affinity with galactose (Gal),N-acetylgalactosamine (GalNAc) include Agaricus bisporus lectin (ABA), Dolichos biflorus lectin (DBA), Erythrina cristagalli lectin (ECA), Phaseolus vulgaris lectin (PHA-E4, PHA-P), peanut lectin (PNA), soybean lectin (SBA), Batihin a purpurea lectin (BPL) and Ricinus communis lectin (RCA 120). Examples of lectins having affinity with mannose (Man) include concanavalin A (ConA), Lens culinaris lectin (LCA) and Pisum sativum lectin (PSA). Examples of lectins having affinity with fucose (Fuc) include Aleuria aurantia. lectin (AAL), Lens culinaris lectin (LCA), lotus lectin (Lotus), Pisum satiyumlectin (PSA), Ulex europaeus lectin (UEA), Lotus corniculatus lectin (LTA), Narcissus pseudonarcissus lectin (NPA), Vicia faha lectin (VFA), Aspergillus oryzae lectin (AOL), Pholiota squarrosa lectin (PhoSL), Pholiota terrestris lectin (PTL), Stropharia rugosoannulata lectin (SRL), Naematoloma sublateritium lectin (NSL). Lepista sordida lectin (LSL) and Amanita muscaria lectin (AML). Above all, the PhoSL, PTL, SRL NSL, LSL and AML are advantageous for detecting a glycoprotein for which the presence of the α→6 fucose is associated with diseases, because they specifically bind only to the α→6 fucose. Examples of lectins having affinity with N-acetylglucosamine (GlcNAc) include Datura stramonium lectin (DSA), pokeweed lectin (PWM), wheat germ lectin (WGA), Griffonia simplicifolia lectin-II (GSL-11) and Psathyrella velutina Lectin (PVL). Examples of lectins having affinity with sialic acid (Sia) include Maackia amurensis lectin (MAM), Sambucus sieboldiana lectin (SSA), wheat germ lectin (WGA), Agrocybe cylindracea lectin (ACG), Trichosanthes japonica lectin (TJA-I). Psathyrella velutina lectin (PVL) and Sambucus nigra lectin (SNA-I).

The sugar-binding compound and/or the probe for detecting the sugar-binding compound are preferably labeled with a labeling means known in the art. Examples of the labeling means may include: an enzyme such as horseradish peroxidase (HRP), alkaline phosphatase (AP), β-D-galactosidase, glucose oxidase and glucose-6-phosphate dehydrogenase; a fluorescent compound such as fluorescein isothiocyanate (FITC), tetramethylrhodamine B isothiocyanate (TRITC), rhodamine and CyDye; a radioactive substance such as ¹²⁵I, ³H and ^{N}C; a metal colloid such as gold sol, silver sot and platinum sol; a synthetic latex such as polystyrene latex colored with a pigment; biotin; and digoxigenin. The probes for detecting the sugar-binding compound may be used either alone or in combination of two or more kinds.

When the labeling means is an enzyme, a chromogenic substrate is used to measure an enzyme activity. The substrate for the horseradish peroxidase (HRP) includes 3,3′,5,5′-tetramethylbenzidine (TMB), 2,2′-azino-di[3-ethylbenzthiazoline sulfonic acid] diammonium salt, 5-aminosalicylic acid, or o-phenylenediamine (OPD). The substrate for the alkaline phosphatase includes p-nitrophenyl phosphate (PNPP) or 4-methylumbellifervi phosphate. The substrate for the β-D-galactosidase is exemplified by o-nitrophenol-β-D-galactopyranoside.

The labeling means can be bound to the sugar-binding compound or the probe for detecting the sugar-binding compound in accordance with a conventional method. In particular, bond via a streptavidin (or avidin)-biotin system is preferable from the viewpoint of increasing the sensitivity.

The glycoprotein is reacted with the sugar-binding compound by exposing the immobilized glycoprotein to a solution containing the sugar-binding compound. The method of the present invention is characterized in that the pH level in at least one step selected from among the group of steps consisting of the reaction step of the glycoprotein with the sugar-binding compound, and the treatment steps subsequent thereto is adjusted to the specific alkaline pH range. That is, in the present invention, it is important to adjust the pH level under an environment of the coexistin2 glycoprotein and sugar-binding compound to the above specific pH range. Specifically, a pH level of the solvent in a sugar-binding compound reaction step for reacting a glycoprotein with a sugar-binding compound to obtain a glycoprotein-sugar-binding compound conjugate, a pH level of a washing solution in a washing step for washing the glycoprotein-sugar-binding compound conjugate, a pH level of a solvent in a probe reaction step for reacting the glycoprotein-sugar-binding compound conjugate with a secondary probe and the subsequent probes, a pH of a washing solution for washing the glycoprotein-sugar-binding compound conjugate after the probe reaction, are adjusted.

The lower limit of the pH level of the solvent is more than 8.5. If the pH level is 8.5 or less, the S/N ratio of the glycoprotein-sugar-binding compound conjugate may not be improved. The lower limit of the pH level is preferably 8.6 or more, more preferably 8.8 or more, still more preferably 9.0 or more. Conversely, the upper limit of the pH level of the solvent is less than 11.0. If the pH level is 11.0 or more, the S/N ratio of the conjugate may not be improved. The upper limit of the pH level is preferably 10.5 or less.

The pH level is adjusted with an alkaline solution, preferably an alkaline buffer. Examples of the buffer include a glycine-sodium hydroxide (NaOH) buffer; a carbonate-bicarbonate buffer; a Good's buffer such as TAPS, Tricine, Bicine, CHES, CAPSO and CAPS; a sodium borate buffer; an ammonium chloride buffer; a broad-range butler such as Britton-Robinson butler; and the like. One or more selected from the glycine-NaOH buffer, the carbonate-bicarbonate buffer and the TAPS butler are preferred, and one or more selected from the glycine-NaOH buffer and the TAPS buffer are more preferable. Preparation of these buffers is based on conventionally known methods.

After the above reaction, the glycoprotein is detected by detecting the reacted sugar-binding compound. The assay method for the sugar-binding compound is not particularly limited, and a method well known to those skilled in the art can be used. Examples of the assay method include: a method for detecting color development, luminescence, fluorescence of an enzyme or the like, such as lectin ELISA (direct adsorption method, sandwich method, competition method) and lectin staining; a method for detecting evanescent waves, such as glycan arrays and lectin arrays; a method for detecting a mass change, such as a crystal oscillator microbalance method and a surface plasmon resonance method; and the like. The surface plasmon resonance method is convenient because a mass of the glycoprotein immobilized on a support and an amount of a detected lectin bound to a glycoprotein can be simultaneously measured by a multistage approach.

Several representative assay methods will be roughly explained below. In the lectin ELISA (direct adsorption method), a solution containing a glycoprotein is added to an ELISA plate and immobilized (solidification). Subsequently, a biotin-labeled lectin is added to react the glycan with the lectin (lectin reaction, primary reaction). An HRP-labeled streptavidin solution is added as a secondary labeled compound to react the biotin with the streptavidin (probe reaction, secondary reaction). Subsequently, a chromogenic substrate for the HRP is added to develop color, and a coloring intensity is measured with an absorptiometer. In at least one of the lectin reaction and the steps subsequent thereto, the pH level is adjusted to an alkaline pH range defined in the present invention. A calibration curve is previously graphed with a known concentration of standard sample, so that the glycan can also be quantitatively determined.

In the lectin ELISA (sandwich method), an antibody capable of binding to a glycoprotein (antigen) is added to a plate or a microplate, and the antibody is immobilized on the plate or the like. Subsequently, a specimen containing a glycoprotein (such as serum) is added to react the antibody with the glycoprotein (specimen reaction). Subsequently, a biotin-labeled lectin is added to react the glycan with the lectin (lectin reaction), An HRP-labeled streptavidin solution is added as a secondary labeled compound to react the biotin with the streptavidin (probe reaction). Subsequently, a chromogenic substrate for the HRP is added to develop color, and a coloring intensity is measured with an absorptiometer. In at least one of the lectin reaction and the steps subsequent thereto, the pH level is adjusted to an alkaline pH range defined in the present invention. A calibration curve is previously graphed with a known concentration of standard sample, so that the glycan can also be quantitatively determined.

In accordance with the method of the present invention, the detection sensitivity for the glycoprotein is improved, contributing to improvement of the diagnostic accuracy for diseases associated with change of the glycan. Examples of diseases for which a galactose residue may be a diagnostic indicator include chronic rheumatoid arthritis, liver cancer, myeloma and the like. Examples of diseases for which a mannose residue may be a diagnostic indicator include rectal cancer and the like. Examples of diseases for which a fucose residue may be a diagnostic indicator include colon cancer, pancreatic cancer, liver cancer and the like. Examples of diseases for which an N-acetylglucosamine residue may be a diagnostic indicator include idiopathic normal pressure hydrocephalus, liver cancer and the like. Examples of diseases for which a sialic acid residue may be a diagnostic indicator include Alzheimer's disease, cardiovascular disease, alcoholism, IgA nephropathy, liver cancer, prostate cancer, ovarian cancer, myocardial infarction, fibrosis, pancreatitis, diabetes, glycoprotein-glycan transfer deficiency, and the like.

### EXAMPLES

Hereinafter, the present invention will be described in more detail with reference to Examples of the present invention. However, the present invention is not limited to the following Examples.

The reagents for use were obtained or prepared as shown below.
<Phosphate buffered saline (pH 7.4) (PBS)>

5.75 g of disodium hydrogenphosphate, 1.0 g of potassium dihydrogenphosphate, 1.0 g of potassium chloride and 40.0 g of sodium chloride were dissolved in 5 L of water to obtain a PBS.
<0.6 M tris-hydrochloric acid buffer (pH 9.0)>

7.3 g of tris-hydroxymethylaminomethane was dissolved in about 80 mL of water, to which 6 N hydrochloric acid was added to adjust the pH level o 9.0, and the volume was further increased to 100 mL with water.
<50 mM glycine-sodium hydroxide (glycine-NaOH) buffer (pH 8.5 to 11.0)>

375.4 mg of glycine was dissolved in about 80 mL of water, to which 5 N sodium hydroxide was added to adjust the pH level to 8.5, and the volume was further increased to 100 mL with water. Similarly, buffers at pH 9.0, pH 9.5, pH 10.0, pH 10.5 and pH 11.0 were prepared.
<50 mM carbonate-bicarbonate buffer (pH 8.6 to 11.0)>

1.06 g of sodium carbonate was dissolved in 200 mL of water, and 0.84 g of sodium hydrogencarbonate was dissolved in 200 mL of water. The sodium carbonate solution and the sodium hydrogencarbonate solution were mixed to prepare buffers at pH 8.6, pH 9.0, pH 9.5, pH 10.0, pH 10.5 and pH 11.0.
<50 mM TAPS buffer (pH 8.5 to 11.0)>

1,22 g of TAPS was dissolved in about 80 mL of water, to which 5 N sodium hydroxide was added to adjust the pH level to 8.5, and the volume was further increased to 100 mL with water. Similarly, buffers at pH 9,0, pH 9,5, pH 10,0, pH 10.5 and pH 11.0 were prepared.
<1.2 M glycine-hydrochloric acid buffer (pH 3.3)>

9.00 g of glycine was dissolved in about 80 mL of water, to which 6N hydrochloric acid was added to adjust the pH level to 3.3, and the volume was further increased to 100 mL with water,
<PBS-T (pH 7.4)>

2.5 mL of polyoxyethylene (20) sorbitan monolaurate (trade name: Tween 20, from Nacalai Tesque, Inc.) was dissolved in 5 L of PBS to obtain a PBS solution of 0.05% Tween 20 (hereinafter referred to as PBS-T).
<Washing Solution for alkaline phosphatase>

10 mL of Wash Solution (20×) (from Kirkegaard & Perry Laboratories. Inc.) was mixed with 90 mL of water.
<1% BSA/PBS>

1 g of bovine serum albumin (BSA, from Sigma-Aldrich Co. LLC) was dissolved in 100 mL of PBS to obtain a PBS solution of 1% BSA (hereinafter referred to as 1% BSA/PBS).
<0.1% BSA/PBS>

100 mg of bovine serum albumin (BSA, from Sigma-Aldrich) was dissolved in 100 mL of PBS to obtain a PBS solution of 0.1% BSA (hereinafter referred to as 0.1% BSA/PBS), p0 <Pepsin Solution>

A pepsin (derived from a swine gastric mucosa, from Sigma-Aldrich Co. LLC) was dissolved in a 1.2 M glycine-hydrochloric acid buffer (pH 3.3) so that the concentration was 1500 U/mL.
<Human Serum>

A human serum from SARL Biopredic International (product name: Human True A serum, Pool of Donors, model number: SER 019A0503634) was used.
<Glycoprotein Solution>

Each of the following glycoproteins was dissolved in PBS so that the concentration was 1 mg/mL, to obtain a glycoprotein solution.
Ovalbumin (OVA, from Sigma-Aldrich Co. LLC),
Fetuin (FET, from Sigma-Aldrich Co., LLC),
Asialofetuin (aFET, from Sigma-Aldrich Co. LLC),
Thyroglobulin (TG, from Scipac Ltd.),
Fucosylated (t-fetoprotein (fAFP) or fucosylated haptoglobin (fHP)
were prepared in accordance with the following method.
<Preparation of fAFP Solution>

A liver cancer cell line (HepG 2, obtained from Institute of Physical and Chemical Research) was cultured in accordance with a conventional method to obtain a culture supernatant. 1000 mL of the culture supernatant was concentrated. to 1 mL with an ultrafiltration filter (product name: VIVA SPIN 20-10 K, from Sartorius AG). The concentrate was added to 0.5 ml of a gel on which an anti-AFP antibody had been immobilized to NHS-activated Sepharose 4 Fast Flow (from GE Healthcare). They were mixed at room temperature every 10 minutes, and after 1 hour, the solution containing the gel was added to a 0.45 μm filter tube (from Millipore Corp.), centrifuged at 400×g and 4° C. for 5 minutes, and the filtrate was discarded. Then, 200 μL of PBS was added, centrifuged at 400×g and 4° C. for 5 minutes, and the filtrate was discarded. This manipulation was repeated twice. Subsequently, 200 μL of Elution Buffer (100 mM glycine, 0.5 M NaCl, pH 3.0) was added, centrifuged at 400×5 and 4° C. for 5 minutes, and the filtrate was recovered. This manipulation was repeated twice. A solution obtained by combining these solutions was neutralized with 3 N NaOH, then 600 μL of PBS was added to obtain a fAFP solution.
<Preparation of fHP Solution>

A fHP solution was obtained in the same manner as the preparation of the above fAFP solution except that a gel on which an anti-haptoglobin antibody had been immobilized instead of the anti-AFP antibody was used.
<Anti-AFP Antibody>

An antibody under the following trade name was obtained. Anti-human AFP monoclonal antibody (mouse) (from Mikuri Immunolab K. K.)
<Deglycosylated Anti-AFP Antibody>

The anti-AFP antibody was deglycosylated in accordance with the method described in Non-Patent Document 1.
<Anti-PhoSL Antibody>

A polyclonal antibody was prepared using a PhoSL as an immunogen in accordance with a known method. An IgG fraction was purified from an antiserum using a protein G solid support.
<Biotin-Labeled Anti-PhoSL Antibody>

A biotinylating reagent (model number: B2643, from Sigma-Aldrich Co. LLC) was dissolved in dimethylsulfoxide, which was added to the above-described anti-PhoSL antibody and reacted. The solvent in the reaction solution was substituted by PBS through ultrafiltration (50 K membrane, from Millipore Corp.) to obtain a biotin-labeled anti-PhoSL antibody solution.
<Lectin>

A Pholiota squarrosa lectin (PhoSL) was purified from Pholiota squarrosa in accordance with the method described in Non-Patent Document 1. A Pholiota terrestris lectin (PTL) was purified from Pholiota terrestris by the same procedure as in the purification of the Pholiota squarrosa lectin. A Stropharia rugosoannulata lectin (SRL) and a Naematoloma sublateritium lectin (NSL) were purified in accordance with the method described in Japanese Patent No. 4514163.
<Biotin-Labeled Lectin>

For a wheat germ lectin (WGA), a Ricinus communis lectin (RCA 120), a Sambucus sieboldiana lectin (SSA) and an Agaricus bisporus lectin (ABA), biotin-labeled lectins from J-OIL MILLS, Inc. were used. The PhoSL, PTL, SRL and NSL were labeled with biotin in accordance with the method described in Non-Patent Document 1, Each of the biotin-labeled lectins was prepared so that the concentration was 1 mg/mL PBS, stored, and at the time of use, it was diluted to an appropriate concentration.

As coloring reagents, a horseradish peroxidase (HRP)-labeled streptavidin (from Kirkegaard & Perry Laboratories, Inc.), a chromogenic substrate for the HRP (trade name:IMB Peroxidase substrate system, from Kirkegaard & Perry Laboratories. Inc.), an alkaline phosphatase (AP)-labeled streptavidin (from Kirkegaard & Perry Laboratories. Inc.), and a chromogenic substrate for AP (trade name: BluePhos®, Microwell Phosphatase Substrate System, from Kirkegaard & Perry Laboratories, Inc.) were prepared.

As coloring reaction-terminating liquids, a reaction-terminating liquid for HRP (1 M phosphoric acid/water) and a reaction-terminating liquid for AP (APStop™ Solution (10×) (from Kirkegaard & Perry Laboratories, Inc.), prepared by mixing 10 mL thereof with 90 mL of water) were prepared.

### Examples 1 to 3

### Measurement of TG by Lectin ELISA (Direct Adsorption Method) (I)

A glycoprotein TG was measured by lectin ELISA (direct adsorption method). Specific steps are shown below
(1) Immobilization of TG

ATG solution (1 mg/mL) was diluted to 5 μg/rat, with PBS. 25 μt, of this diluted solution was added to each well of a 96-well microliter plate and allowed to stand at 4° C. overnight, and then the additive solution was discarded.
(2) Washing

150 μL of PBS-T was added to the well, and the additive solution was discarded.
(3) Blocking

50 μL of 1% BSA/PBS was added to the well and allowed to stand at 37° C. for 60 minutes, and then the additive solution was discarded.
(4) Washing

150 μL was PBS-T was added to the well, and the additive solution was discarded. This manipulation was repeated twice in total.
(5) Specimen Reaction (Addition of Human Serum)

Glycoproteins such as TG are normally contained in biological samples such as serum and blood. These samples contain various substances in addition to the desired glycoprotein. These substances act as noise sources during measurement of a glycoprotein. Therefore, in order to simulate the noise sources during measurement of the glycoprotein, human serum was additionally added to the well. Specifically, 25 μL of human serum was added to the well and allowed to stand at room temperature for 30 minutes, and then the additive solution was discarded.
(6) Washing

150 μL of PBS-T was added to the well, and the additive solution was discarded. This manipulation was repeated three times in total.
(7) Primary Reaction (Reaction of Glycoprotein with Lectin)

The biotin-labeled PhoSL was diluted to 0.25 μg/mL with buffers shown in Table 1. 25 μL of this lectin solution was added to the well and allowed to stand at room temperature for 30 minutes, and then the additive solution was discarded.
(8) Washing

150 μL of PBS-T was added to the well, and the additive solution was discarded. This manipulation was repeated three times in total.
(9) Secondary Reaction (HRP-Labeled Streptavidin Reaction)

The HRP-labeled streptavidin was diluted to 0.04 μg/mL with PBS. 25 μL of this solution was added to the well and allowed to stand at room temperature for 30 minutes, and then the additive solution was discarded.
(10) Washing

150 μL of PBS-T was added to the well, and the additive solution was discarded. This manipulation was repeated four times in total.
(II) Coloring Reaction

25 μL of chromogenic substrate for HRP was added to the well and allowed to stand at room temperature for 10 minutes.
(12) Termination of Reaction

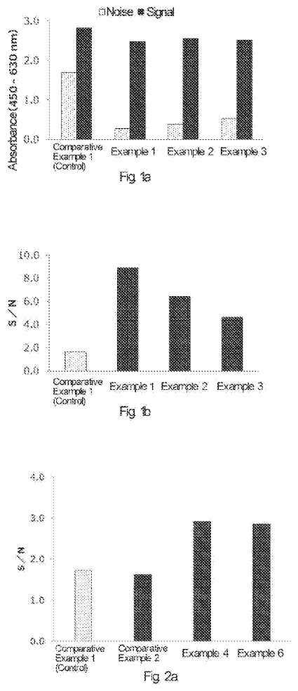

25 μL of reaction-terminating liquid (1 M phosphate/water) was added to terminate the coloring reaction. Absorbance at 450 nm and 630 nm was measured using a plate reader (product name: POWERSCAN® HT, from BioTek Instruments, Inc.). A value obtained by subtracting the absorbance at 630 nm from the absorbance at 450 nm in the case with the glycoprotein was taken as a signal value: Absorbance_{TG(+)}. Similarly, a value obtained by subtracting the absorbance at 630 nm from the absorbance at 450 nm in the case without the glycoprotein was taken as a noise value: Absorbance_{TG(-)}. As shown in Formula (1), the S/N ratio was determined by dividing the signal value in the case with the glycoprotein by the noise value in the case without the glycoprotein. The results are shown in Table 1, and FIGS. 1a and 1b.

[Equation 1]

S/N=Absorbance_{TG(+)}/Absorbance_{TG(-)} (1)

TABLE 1

| | | | | | | | S/N |
|---|---|---|---|---|---|---|---|
| | Glycoprotein | Lectin | Buffer | pH | Absorbance_{TG (-)} | Absorbance_{TG (+)} | ratio |
| Comparative | TG | PhoSL | PBS | 7.4 | 1.702 | 2.828 | 1.7 |
| Example 1 | | | | | | | |
| Example 1 | | | Glycine- | 10.0 | 0.279 | 2.480 | 8.9 |
| | | | Sodium | | | | |
| | | | hydroxide | | | | |
| Example 2 | | | Carbonic acid- | 10.0 | 0.399 | 2.565 | 6.4 |
| | | | Bicarbonate | | | | |
| Example 3 | | | TAPS | 10.0 | 0.543 | 2.514 | 4.6 |

As in Comparative Example 1, as a result of detecting a serum containing a glycoprotein-lectin conjugate in a buffer at an pH level within a neutral range, the signal value is as high as 2.828, but the noise value is also as high as 1.702. The S/N ratio in Comparative Example 1 is 1.7. On the other hand, as in Examples 1 to 3, the solvent in the reaction of the glycoprotein TG with the lectin is adjusted to have an alkaline pH level, so that the noise value attributed to the serum is remarkably decreased while maintaining the signal value attributed to the glycoprotein at a high level (Table 1 and FIG. 1a). As a result, the detection sensitivity of the glycoprotein-lectin conjugate represented by the S/N ratio is markedly increased by 4.6 to 8.9 times (Table 1 and FIG. 1b).

### Examples 4 to 71

### Measurement of TG by lectin ELISA (Direct Adsorption Method) (II)

In Example 1, a solvent at an alkaline pH level was used during (7) the reaction of the glycoprotein with the lectin, but the conditions were changed as below in Examples 4 to 7.

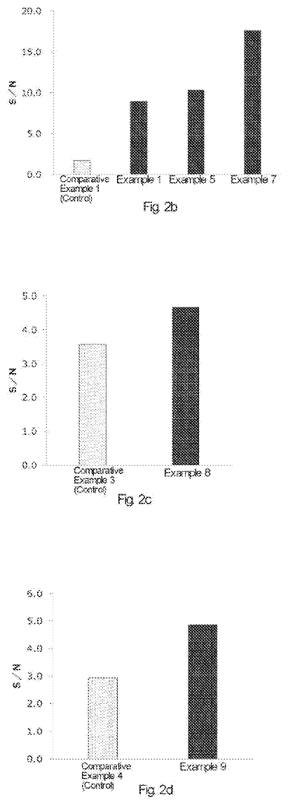

In Example 4, the TG was measured by the same procedure as in Comparative Example 1, except that the (8) washing after the primary reaction was changed from the PBS-T×3 washings to PBS-T×1, glycine-sodium hydroxide buffer (pH 10.0)×2 and PBS-T×1 washings (4 washings in total) in Comparative Example 1. The results are shown in Table 2 and FIG. 2a.

In Example 5, the TG was measured by the same procedure as in Comparative Example 1, except that the solvent in the (9) secondary reaction was changed from the PBS (pH 7.4) to a glycine-sodium hydroxide buffer (pH 10.0) in Comparative Example 1. The results are shown in Table 2 and FIG. 2b.

In Example 6, the TG was measured by the same procedure as in Comparative Example 1, except that the (10) washing after the secondary reaction was changed from the PBS-T×3 washings to PBS-T×1, glycine-sodium hydroxide buffer (pH 10.0)×2 and PBS-T×1 washings (4 washings in total) in Comparative Example 1. The results are shown in Table 2 and FIG. 2a.

In Example 7, the TG was measured by the same procedure as in Comparative Example 1, except that the solvent in the (7) primary reaction and the solvent in the (9) secondary reaction were changed to a glycine-sodium hydroxide buffer (pH 10.0) in Comparative Example 1. The results are shown in Table 2 and FIG. 2b.

In Comparative Example 2, the TG was measured by the same procedure as in Comparative Example 1, except that the (6) washing after the specimen reaction was changed from the PBS-T×3 washings to PBS-T×1, glycine-sodium hydroxide buffer (pH 10.0)×2 and PBS-T×1 washings (4 washings in total) in Comparative Example 1. The results are shown in Table 2.

In Comparative Example 3, the TG was measured by the same procedure as in Comparative Example 1, except that the probe in the (9) secondary reaction was changed to an AP-labeled streptavidin (0.5 μg/ml), and the washing solution, the chromogenic substrate and the coloring reaction-terminating liquid were changed to those for the alkaline phosphatase depending on the enzyme in Comparative Example 1. In Example 8, the TG was measured by the same procedure as in Comparative Example 3 except that the solvent in the second reaction was changed to a glycine-sodium hydroxide buffer (pH 10.0) in Comparative Example 3. These results are shown in Table 2 and FIG. 2c.

In Comparative Example 4, the TG was measured in accordance with Comparative Example 1, except that a PhoSL (unlabelled) was used for the primary probe, a biotin-labeled anti-PhoSL antibody was used for the secondary probe and an HRP-labeled streptavidin was used for the tertiary probe. All probe reactions were carried out in PBS. In Example 9, the TG was measured by the same procedure as in Comparative Example 4, except that the solvent in the secondary reaction using the secondary probe was changed to a glycine-sodium hydroxide buffer (pH 10.0). These results are shown in Table 2 and FIG. 2d.

TABLE 2

| | | Primary | Secondary | Tertiary | | | Step of applying | | | S/N |
|---|---|---|---|---|---|---|---|---|---|---|
| | Glycoprotein | probe | probe | probe | Buffer | pH | alkaline pH range | Absorbance_{TG (-)} | Absorbance_{TG (+)} | ratio |
| Comparative | TG | Biotin- | HRP-labeled | — | PBS | 7.4 | — | 1.702 | 2.828 | 1.7 |
| Example 1 | | labeled | streptavidin | | | | | | | |
| Comparative | | PhoSL | | | Glycine- | 10.0 | (6) Washing | 1.780 | 2.874 | 1.6 |
| Example 2 | | | | | Sodium | | solution after | | | |
| | | | | | | | specimen reaction | | | |
| Example 1 | | | | | hydroxide | 10.0 | (7) Solvent in | 0.279 | 2.480 | 8.9 |
| | | | | | | | primary reaction | | | |
| Example 4 | | | | | | 10.0 | (8) Washing | 0.987 | 2.868 | 2.9 |
| | | | | | | | solution after | | | |
| | | | | | | | primary reaction | | | |
| Example 5 | | | | | | 10.0 | (9) Solvent in | 0.107 | 1.098 | 10.3 |
| | | | | | | | secondary | | | |
| | | | | | | | reaction | | | |
| Example 6 | | | | | | 10.0 | (10) Washing | 0.963 | 2.753 | 2.9 |
| | | | | | | | solution after | | | |
| | | | | | | | secondary | | | |
| | | | | | | | reaction | | | |
| Example 7 | | | | | | 10.0 | (7) Solvent in | 0.032 | 0.563 | 17.9 |
| | | | | | | | primary reaction, | | | |
| | | | | | | | and | | | |
| | | | | | | | (9) Solvent in | | | |
| | | | | | | | secondary | | | |
| | | | | | | | reaction | | | |
| Comparative | | | AP-labeled | | PBS | 7.4 | — | 0.593 | 2.120 | 3.6 |
| Example 3 | | | streptavidin | | | | | | | |
| Example 8 | | | | | Glycine- | 10.0 | (9) Solvent in | 0.076 | 0.354 | 4.7 |
| | | | | | Sodium | | secondary | | | |
| | | | | | hydroxide | | reaction | | | |
| Comparative | | PhoSL | Biotin- | HRP- | PBS | 7.4 | — | 0.795 | 2.338 | 2.9 |
| Example 4 | | | labeled anti- | labeled | | | | | | |
| Example 9 | | | PhoSL | streptavidin | Glycine- | 10.0 | (9) Solvent in | 0.108 | 0.526 | 4.9 |
| | | | antibody | | Sodium | | secondary | | | |
| | | | | | hydroxide | | reaction | | | |

In Comparative Example 1, as a result of conducting the lectin reaction and the subsequent washing at pH 7.4, the SIN ratio was 1.7. Even when the pH level in the washing after the (6) specimen reaction was changed to an alkaline level of pH 1 0.0 as in Comparative Example 2, the S/N ratio was not improved. From this, it can be seen that the S/N ratio is not improved only by changing the environment of the glycoprotein alone to the alkaline pH level.

On the other hand, as in Examples 1,4, 5, 6, 7, 8 and 9, the pH level of any of the solvent during the primary reaction, the washing solution after the primary reaction, the solvent during the secondary reaction or the washing solution after the secondary reaction was changed to the pH range defined in the present invention, and then the S/N ratio was improved. That is, the S/N ratio is improved by changing the pH level under the environment of the coexisting glycoprotein and lectin to an alkaline pH level (FIGS. 2a and 2b). In particular, it is preferable to adjust the solvents during the primary reaction and/or secondary reaction so as to have alkaline pH levels, from the viewpoint of remarkably increasing the S/N ratio (FIG. 2b).

### Examples 10 to 22

### Measurement of fAFP by Lectin ELISA (Sandwich Method) (I)

An fAFP glycoprotein was measured by lectin ELISA (sandwich method). In this example, the glycoprotein was processed into glycopeptides before measurement. Specific steps are shown below.
1. Protease Treatment

Protease treatment of the glycoprotein fAFP was carried out using pepsin. Specifically,the fAFP solution was added to a human serum so that a concentration of the fAFP solution was 400 ng/mL. Next, 2.5 mL of the fAFP-added serum solution and 1.25 mL of the pepsin solution were mixed, stirred, and then allowed to stand at 37° C. for 30 minutes. 1.25 mL of 0.6 M tris-hydrochloric acid buffer (pH 9.0) was added to this solution to terminate the proteolytic reaction, and a serum solution of a pepsin-treated fAFP (fAFP (+)) at a concentration of 200 ng/mL was obtained. As a blank, a pepsin-treated serum solution containing no fAFP (fAFP (−)) was prepared by treating the human serum with pepsin without adding the fAFP.
2. Detection of glycoprotein

The fAFP (+) or fAFP (−) obtained above was detected by a sandwich ELISA using a biotin-labeled PhoSL diluted with the buffers shown in Table 3. The sandwich ELISA was in accordance with the method described in Non-Patent Document 1. Specifically, the test was carried out by the following procedure.
(1) Antibody Immobilization

A deglycosylated anti-AFP antibody was diluted to 5 μg/mL with PBS, 25 μL of this solution was added to each well of a 96-well microtiter plate, allowed to stand at 4° C. overnight, and then the additive solution was discarded.
(2) Washing

150 μL of PBS-T was added to the well, and the additive solution was discarded.
(3) Blocking

50 μL of 1% BSA/PBS was added to the well and allowed to stand at 37° C. for 60 minutes, and then the additive solution was discarded.
(4) Washing

150 μL of PBS-T was added to the well, and the additive solution was discarded. This manipulation was repeated twice in total.
(5) Specimen Reaction

25 μL of the fAFP (+) or the fAFP (−) was added to the well and allowed to stand at room temperature for 30 minutes, and then the additive solution was discarded.
(6) Washing

150 uL of PBS-T was added to the well, and the additive solution was discarded. This manipulation was repeated three times in total.
(7) Primary Reaction (Reaction of Glycoprotein with Lectin)

The biotin-labeled PhoSL was diluted to 0.25 μg/mL with buffers shown in Table 3. 25 μL of this lectin solution was added to the well and allowed to stand at room temperature for 30 minutes, and then the additive solution was discarded.
(8) Washing

150 μL of PBS-T was added to the well, and the additive solution was discarded. This manipulation was repeated three times in total.
(9) Secondary Reaction (HRP-Labeled Streptavidin Reaction)

The HRP-labeled streptavidin was diluted to 0.04 μg/mL with PBS. 25 μL of this solution was added to each well and allowed to stand at room temperature for 30 minutes, and then the additive solution was discarded.
(10) Washing

150 μL of PBS-T was added to the well, and the additive solution was discarded. This manipulation was repeated three times in total.
(11) Coloring Reaction

25 μL of chromogenic substrate for HRP was added to the well and allowed to stand at room temperature for 10 minutes.
(12) Termination of Reaction

25 μL of reaction-terminating liquid (1 M phosphate/water) was added to the well to terminate the coloring reaction. Absorbance at 450 nm and 630 nm was measured using the plate reader. A value obtained by subtracting the absorbance value at 630 nm from the absorbance value at 450 nm in the case with the fAFP (+) was taken as a signal value (Absorbance_{fAFP(-)}). In the same manner, a value in the case with the fAFP (−) was determined as a noise value (Absorbance_{fAFP(-)}). Subsequently, the S/N ratio of the fAFP was determined from Equation (2).

[Equation 2]

S/N=Absorbance_{fAFP(+)}/Absorbance_{fAFP(-)} (2)

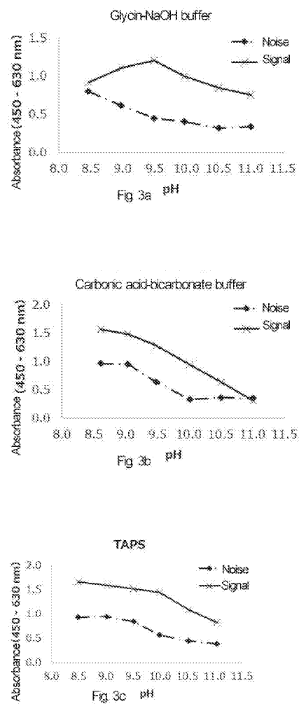

The results of the cases of using various buffers for the solvent during the primary reaction are shown in Table 3 and FIGS. 3a to 3d.

TABLE 3

| | | | | | | | S/N |
|---|---|---|---|---|---|---|---|
| | Glycoprotein | Lectin | Buffer | pH | Absorbance_{fAFP (-)} | Absorbance_{fAFP (+)} | ratio |
| Comparative | fAFP | PhoSL | PBS | 7.4 | 1.464 | 1.760 | 1.2 |
| Example 5 | | | | | | | |
| Comparative | | | Glycine- | 8.5 | 0.803 | 0.910 | 1.1 |
| Example 6 | | | Sodium | | | | |
| Example 10 | | | hydroxide | 9.0 | 0.620 | 1.106 | 1.8 |
| Example 11 | | | | 9.5 | 0.451 | 1.203 | 2.7 |
| Example 12 | | | | 10.0 | 0.401 | 1.000 | 2.5 |
| Example 13 | | | | 10.5 | 0.319 | 0.848 | 2.7 |
| Reference | | | | 11.0 | 0.337 | 0.752 | 2.2 |
| Example 1 | | | | | | | |
| Example 14 | | | Carbonic | 8.6 | 0.968 | 1.563 | 1.6 |
| Example 15 | | | acid- | 9.0 | 0.953 | 1.485 | 1.6 |
| Example 16 | | | Bicarbonate | 9.5 | 0.643 | 1.279 | 2.0 |
| Example 17 | | | | 10.0 | 0.333 | 0.948 | 2.8 |
| Example 18 | | | | 10.5 | 0.364 | 0.639 | 1.8 |
| Comparative | | | | 11.0 | 0.349 | 0.311 | 0.9 |
| Example 7 | | | | | | | |
| Reference | | | TAPS | 8.5 | 0.936 | 1.660 | 1.8 |
| Example 2 | | | | | | | |
| Example 19 | | | | 9.0 | 0.945 | 1.582 | 1.7 |
| Example 20 | | | | 9.5 | 0.844 | 1.518 | 1.8 |
| Example 21 | | | | 10.0 | 0.564 | 1.445 | 2.6 |
| Example 22 | | | | 10.5 | 0.443 | 1.083 | 2.4 |
| Reference | | | | 11.0 | 0.382 | 0.818 | 2.1 |
| Example 3 | | | | | | | |

From the results in Table 3 and FIGS. 3a to 3d, it was proved that even when the glycoprotein was changed from the TG to the fAFP, the detection sensitivity represented by the S/N ratio of the glycoprotein was improved, and that even when the glycoprotein was fragmented into glycopeptides, the S/N ratio was improved.

The S/N ratio in Comparative Example 5 using a conventional PBS at pH 7.4 as the solvent for the primary reaction was 1.2, and it was confirmed that when changing the buffer to the glycine-sodium hydroxide buffer at an alkaline pH level, the S/N ratio was improved at a pH level within a range of more than 8.5 to 11 (Examples 10 to 13 and Reference Example 1), that when changing the buffer to the carbonate-bicarbonate buffer, the S/N ratio was improved at a pH level within a range of 8.6 to less than 11.0 (Examples 14 to 18), and that when changing the buffer to the TAPS buffer, the S/N ratio was improved at a pH level within a range of 8.5 to 11.0 (Examples 19 to 22, and Reference Examples 2 and 3).

The above results suggest that the detection sensitivity for the glycoprotein is improved by adjusting the pH levels of the solvents during the lectin reaction and the steps subsequent thereto to a range of more than 8.5 to less than 11,0, preferably a range of 8.6 to 10.5, more preferably a range of 9.0 to 10.5.

### Example 23

### Measurement of fAFP by Lectin ELISA (Sandwich Method) (II)

An experiment was carried out, in which the immobilization support for the lectin ELISA in Example 12 was changed to microbeads. The specific procedure is described below.
1. Protease Treatment

Protease treatment of the glycoprotein fAFP was carried out in the same manner as in Example 12.
2. Detection of Glycoprotein

The fAFP (+) or fAFP (−) obtained above was detected by a microbeads ELISA (sandwich method) using a biotin-labeled lectin shown in Table 4. Specifically, the test was carried out by the following procedure. Note that a plate-shaped magnet was appropriately used in order to hold the microbeads.
(1) Antibody Immobilization

Microbeads (trade name: Dynabeads (registered trademark) M-280 Tosylactivated, from VERITAS Corporation) and a deglycosylated anti-AFP antibody were prepared so that the density of the antibody was 20 μg/mg beads, and immobilized at 37° C. overnight, and then diluted with a 0.1% BSA/PBS to prepare a 2% bead suspension. 1 μL of the bead suspension was added to each well of a 96-well microtiter plate.
(2) Washing

150 μL of PBS-T was added to the well, and the additive solution was discarded.
(3) Specimen Reaction

25 μL of the fAFP (+) or the fAFP (−) was added to the well, stirred, then allowed to stand at room temperature for 30 minutes, and then the additive solution was discarded.
(4) Washing

150 μL of PBS-T was added to the well, stirred, and then the additive solution was discarded. This manipulation was repeated three times in total.
(5) Primary Reaction (reaction of Glycoprotein with Lectin)

The biotin-labeled PhoSL was diluted to 0.25 μg/mL with buffers shown in Table 4. 25 μL of this lectin solution was added to the well, stirred, then allowed to stand at room temperature for 30 minutes, and then the additive solution was discarded.
(6) Washing

150 μL of PBS-T was added to the well, stirred, and then the additive solution was discarded. This manipulation was repeated three times in total.
(7) Secondary Reaction (HRP-Labeled Streptavidin Reaction)

The HRP-labeled streptavidin was diluted to 0.04 μg/mL with PBS. 25 μL of this solution was added to each well, stirred, then allowed to stand at room temperature for 30 minutes, and then the additive solution was discarded.
(8) Washing

150 μL of PBS-T was added to each well, stirred, and then the additive solution was discarded. This manipulation was repeated three times in total.
(9) Coloring Reaction

25 μL of chromogenic substrate for HRP was added to each well, stirred, and then allowed to stand at room temperature for 10 minutes.
(10) Termination of Reaction

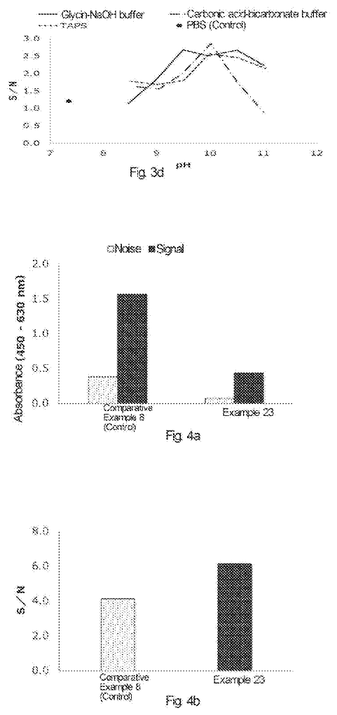

25 μL of reaction-terminating liquid (1 M phosphate/water) was added to the well to terminate the coloring reaction. A signal value and a noise value were measured in the same manner as in Example 12, and the S/N ratio was determined. The results are shown in Table 4, FIGS. 4a and 4b.

TABLE 4

| | | | | | | | S/N |
|---|---|---|---|---|---|---|---|
| | Glycoprotein | Lectin | Buffer | pH | Absorbance_{fAFP (-)} | Absorbance_{fAFP (+)} | ratio |
| Comparative | fAFP | PhoSL | PBS | 7.4 | 0.378 | 1.566 | 4.1 |
| Example 8 | | | | | | | |
| Example 23 | | | Glycine- | 9.5 | 0.071 | 0.436 | 6.1 |
| | | | Sodium | | | | |
| | | | hydroxide | | | | |

In Table 4, the S/N ratio in Comparative Example 8 using a conventionally used PBS at pH 7.4 was 4.1. On the other hand, when using a glycine-sodium hydroxide buffer at pH 9.5, the S/N ratio was improved to 6.1. It is indicated that the method of the present invention does not depend on the type of the solid support.

### Examples 24 to 37

### Measurement of Various Glycoproteins By Lectin ELISA (Direct Adsorption Method)

(1) Immobilization of Glycoprotein

Each of the glycoproteins shown in Table 5 was diluted to 5 μg/mL with PBS. 25 μL of this diluted solution was added to each well of a 96-well microtiter plate, allowed to stand at 4° C. overnight, and then the additive solution was discarded.
(2) Washing

150 μL of PBS-T was added to the well, and the additive solution was discarded.
(3) Blocking

50 82 L of 1% BSA/PBS was added to the well and allowed to stand at 37° C. for 60 minutes, and then the additive solution was discarded.
(4) Washing

150 μL of PBS-T was added to the well, and the additive solution was discarded. This manipulation was repeated twice in total.
(5) Specimen Reaction (Addition Of Human Serum)

25 μL of human serum was added to the well and allowed to stand at room temperature for 30 minutes, and then the additive solution was discarded.
(6) Washing

150 μL of PBS-T was added to the well, and the additive solution was discarded. This manipulation was repeated three times in total.
(7) Primary Reaction (Reaction of Glycoprotein with Lectin)

Each of the biotin-labeled lectins shown in Table 5 was diluted to 0.25 μg/mL with buffers shown in Table 5. 25 μL of this lectin solution was added to the well and allowed to stand at room temperature for 30 minutes, and then the additive solution was discarded.
(8) Washing

150 μL of PBS-T was added to the well, and the additive solution was discarded. This manipulation was repeated three times in total.
(9) Secondary Reaction (HRP-Labeled Streptavidin Reaction)

The HRP-labeled streptavidin was diluted to 0.04 μg/mL with PBS. 25 μL of this solution was added to each well and allowed to stand at room temperature for 30 minutes, and then the additive solution was discarded.
(10) Washing

150 μL of PBS-T was added to each well, and the additive solution was discarded. This manipulation was repeated four times in total.
(11) Coloring Reaction

25 μL of chromogenic substrate for HRP was added to each well and allowed to stand at room temperature for 10 minutes.
(12) Termination of Reaction

25 μL of reaction-terminating liquid (1 M phosphate/water) was added to terminate the coloring reaction. Absorbance at 450 nm and 630 nm was measured using the plate reader. A value obtained by subtracting the absorbance at 630 nm from the absorbance at 450 nm in the case with the glycoprotein was taken as a signal value: Absorbance_{glycoprotein(+)}. Similarly, a value obtained by subtracting the absorbance at 630 nm from the absorbance at 450 nm in the case without the glycoprotein was taken as a noise value: Absorbance_{glycoprotein(-)}. As shown in Formula (3), the S/N ratio was determined by dividing the signal value by the noise value. The results are shown in Table 5.

[Equation 3]

S/N=Absorbance_{glycoprotein(+)}/Absorbance_{glycoprotein(-)} (3)

TABLE 5

| | | | | | | | S/N |
|---|---|---|---|---|---|---|---|
| | Glycoprotein | Lectin | Buffer | pH | Absorbance_{Glycoprotein (-)} | Absorbance_{Glycoprotein (+)} | ratio |
| Comparative | OVA | WGA | PBS | 7.4 | 0.642 | 2.063 | 3.2 |
| Example 9 | | | | | | | |
| Example 24 | | | Glycine- | 10.0 | 0.023 | 0.815 | 35.4 |
| | | | Sodium | | | | |
| | | | hydroxide | | | | |
| Comparative | aFET | RCA120 | PBS | 7.4 | 0.390 | 2.512 | 6.4 |
| Example 10 | | | | | | | |
| Example 25 | | | Glycine- | 10.0 | 0.138 | 2.496 | 18.2 |
| | | | Sodium | | | | |
| | | | hydroxide | | | | |
| Comparative | | ABA | PBS | 7.4 | 1.406 | 2.801 | 2.0 |
| Example 11 | | | | | | | |
| Example 26 | | | Glycine- | 10.0 | 0.305 | 2.754 | 9.0 |
| | | | Sodium | | | | |
| | | | hydroxide | | | | |
| Comparative | FET | SSA | PBS | 7.4 | 2.350 | 2.722 | 1.2 |
| Example 12 | | | | | | | |
| Example 27 | | | Glycine- | 10.0 | 0.539 | 1.262 | 2.3 |
| | | | Sodium | | | | |
| | | | hydroxide | | | | |
| Comparative | | ABA | PBS | 7.4 | 1.406 | 2.800 | 2.0 |
| Example 13 | | | | | | | |
| Example 28 | | | Glycine- | 10.0 | 0.305 | 2.444 | 8.0 |
| | | | Sodium | | | | |
| | | | hydroxide | | | | |
| Comparative | TG | SSA | PBS | 7.4 | 2.350 | 2.719 | 1.2 |
| Example 14 | | | | | | | |
| Example 29 | | | Glycine- | 10.0 | 0.539 | 1.433 | 2.7 |
| | | | Sodium | | | | |
| | | | hydroxide | | | | |
| Comparative | | PhoSL | PBS | 7.4 | 0.376 | 2.332 | 6.2 |
| Example 15 | | | | | | | |
| Example 30 | | | Glycine- | 10.0 | 0.049 | 1.297 | 26.7 |
| | | | Sodium | | | | |
| | | | hydroxide | | | | |
| Comparative | | PTL | PBS | 7.4 | 0.266 | 2.550 | 9.6 |
| Example 16 | | | | | | | |
| Example 31 | | | Glycine- | 10.0 | 0.091 | 1.798 | 19.9 |
| | | | Sodium | | | | |
| | | | hydroxide | | | | |
| Comparative | | SRL | PBS | 7.4 | 1.075 | 2.569 | 7.4 |
| Example 17 | | | | | | | |
| Example 32 | | | Glycine- | 10.0 | 0.082 | 1.970 | 24.2 |
| | | | Sodium | | | | |
| | | | hydroxide | | | | |
| Comparative | | NSL | PBS | 7.4 | 1.501 | 2.439 | 1.6 |
| Example 18 | | | | | | | |
| Example 33 | | | Glycine- | 10.0 | 0.456 | 2.055 | 4.5 |
| | | | Sodium | | | | |
| | | | hydroxide | | | | |
| Comparative | fHP | PhoSL | PBS | 7.4 | 0.376 | 2.132 | 5.0 |
| Example 19 | | | | | | | |
| Example 34 | | | Glycine- | 10.0 | 0.049 | 1.297 | 17.7 |
| | | | Sodium | | | | |
| | | | hydroxide | | | | |
| Comparative | | PTL | PBS | 7.4 | 0.266 | 2.480 | 9.3 |
| Example 20 | | | | | | | |
| Example 35 | | | Glycine- | 10.0 | 0.091 | 1.465 | 16.2 |
| | | | Sodium | | | | |
| | | | hydroxide | | | | |
| Comparative | | SRL | PBS | 7.4 | 1.075 | 2.585 | 2.4 |
| Example 21 | | | | | | | |
| Example 36 | | | Glycine- | 10.0 | 0.082 | 1.715 | 21.0 |
| | | | Sodium | | | | |
| | | | hydroxide | | | | |
| Comparative | | NSL | PBS | 7.4 | 1.501 | 2.456 | 1.6 |
| Example 22 | | | | | | | |
| Example 37 | | | Glycine- | 10.0 | 0.456 | 1.700 | 3.7 |
| | | | Sodium | | | | |
| | | | hydroxide | | | | |

From the results in Table 5, it was proved that the method of the present invention could improve the SA ratio regardless of the types of the glycoprotein and its glycan, and the sugar-binding compound having affinity therewith.

1. A glycoprotein assay method, comprising reacting a glycoprotein with a sugar-binding compound having affinity with a glycan contained in the glycoprotein to detect the reacted sugar-binding compound, wherein
a pH level is adjusted to an alkaline pH range of more than 8.5 to less than 11.0, in at least one step selected from among a group of steps consisting of the reaction step of the glycoprotein with the sugar-binding compound, and treatment steps subsequent thereto.

2. The glycoprotein assay method according to claim 1, wherein the sugar-binding compound is a sugar-binding protein.

3. The glycoprotein assay method according to claim 1, comprising adjusting the pH level in the reaction step of the glycoprotein with the sugar-binding compound to the alkaline pH range.

4. The glycoprotein assay method according to claim 1, wherein the glycoprotein is immobilized to a support.

5. The glycoprotein assay method according to claim 4, wherein the glycoprotein is immobilized to the support via an antibody of the glycoprotein.

6. The glycoprotein assay method according to claim 1, wherein the sugar-binding compound and/or a probe for detecting the sugar-binding compound are labeled.

7. The glycoprotein assay method according to claim 1, wherein the glycan is a complex-type glycan or an O-linked glycan.

8. The glycoprotein assay method according to claim 1, wherein the glycoprotein is one selected from a group consisting of haptoglobin, fucosylated haptoglobin, transferrin, γ-glutamyltranspeptidase, immunoglobulin immunoglobulin A, immunoglobulin M, α1-acidic glycoprotein, α-fetoprotein, fucosylated α-fetoprotein, fibrinogen, human placenta chorionic gonadotropin, carcinoembryonic antigen, prostate-specific antigen, thyroglobulin, fetuin, asialofetuin and ovalbumin.
